(19) United States
(12) Patent Application Publication
(10) Pub. No.: US 2024/0425446 A1
(43) Pub. Date: Dec. 26, 2024

(54) DICHLOROMETHANE-FREE SYNTHESIS OF CYCLOPROPENIMINES

(71) Applicants: International Business Machines Corporation, Armonk, NY (US); The Trustee of Columbia University in the City of New York, New York, NY (US)

(72) Inventors: James L. Hedrick, Pleasanton, CA (US); Luis M. Campos, New York, NY (US); Nathaniel H. Park, San Jose, CA (US); Dino Wu, New York, NY (US)

(21) Appl. No.: 18/340,047

(22) Filed: Jun. 23, 2023

(51) Int. Cl. C07C 249/02; B01D 53/62; C01B 32/60

(52) U.S. Cl. CPC C07C 249/02; B01D 53/62; C01B 32/60; B01D 2252/20436; B01D 2257/504

(57) ABSTRACT

A process of forming a cyclopropenimine (CPI) is disclosed. The process includes obtaining a solution of pentachlorocyclopropane (PCC) dissolved in a first solvent and adding a secondary amine to the solution. The process further includes obtaining precipitated products, including a CPI chloride (CPI−Cl) salt and a secondary amine salt, of a reaction between the secondary amine and the PCC and mixing the precipitated products in a second solvent. The CPI−Cl salt is substantially soluble in the second solvent and the secondary amine salt is substantially insoluble in the second solvent. A CPI composition, an apparatus containing the CPI composition, a process of capturing carbon dioxide (CO₂) with the CPI composition, and a process of generating carbonate with the CPI composition are also disclosed.

### BACKGROUND

The present disclosure relates to small-molecule synthesis and, more specifically, to synthesis of cyclopropenimine compounds.

Cyclopropenimines (CPIs) are a class of strong organic bases, which can act as enantioselective Brønsted base catalysts. CPIs can be used for direct-air-capture of CO₂ by forming adducts with CO₂. Low-energy CO₂ release from the adducts can be accomplished with external stimuli (e.g., mechano- or photochemical activation). Further, CPIs can activate the captured CO₂ for subsequent chemical transformations, which can be followed by recovery of the CPI via basification. The aromatic stabilization and synthetic modularity of CPIs have enabled their use in catalytic transformations such as Michael additions, Mannich reactions, Wittig rearrangements, and ring-opening polymerization. Conjugate acids of CPIs can be utilized as Brønsted acid catalysts in reactions involving additions to oxocarbeniums and hydroamination of alkenes.

Further, CPIs can be synthesized on a large scale from readily available, inexpensive substrates and processed into emulsions, membranes, particles, etc. that may be integrated within CO₂ reactors. However, existing techniques for CPI synthesis typically require use of dichloromethane for solubility reasons. Health risks associated with dichloromethane can limit the application of CPIs, particularly in large-scale processes. Therefore, a need exists for new synthetic methods that can produce CPIs without dichloromethane or other harmful solvents.

### SUMMARY

Various embodiments directed to a process of forming a cyclopropenimine (CPI) are disclosed. The process includes obtaining a solution of pentachlorocyclopropane (PCC) dissolved in a first solvent and adding a secondary amine to the first solution. The process further includes obtaining precipitated products, including a CPI chloride (CPI−Cl) salt and a secondary amine salt, of a reaction between the secondary amine and the PCC and mixing the precipitated products in a second solvent. The CPI−Cl salt is substantially soluble in the second solvent, and the secondary amine salt is substantially insoluble in the second solvent. In some embodiments, the process includes extracting the CPI−Cl salt from the mixture and preparing a solution of the extracted CPI−Cl salt and a primary amine in a third solvent. The process can further include obtaining a tris[amino]cyclopropenium (TAC) salt formed in a reaction between the CPI−Cl salt and the primary amine. The TAC salt may be polymerized in some embodiments. The TAC salt can be neutralized in an alkaline solution to generate the CPI. When the TAC salt has been polymerized, the neutralization can result in formation of a polymer with CPI pendent groups.

An advantage of the process of forming CPIs over existing techniques, especially large-scale synthesis, can be that it does not require dichloromethane (DCM). For example, first solvent may be ethyl acetate, the second solvent may be chloroform, and the third solvent may be toluene. These are readily available solvents that are safer than DCM.

The CPI can have the following structure:

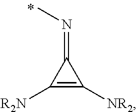

where each R is an organic substituent, and the starred bond is to a carbon atom. The primary amine may be n-butylamine, cyclohexylamine, propylimidazole, N,N-dimethyl-1-propanamine, N,N-dimethyldiethylenetriamine, 2-aminoethanol, 2-amino-1-propanol, 1,2-diaminoethane, 1-hydroxy-2-butylamine, 2-aminopropane-1,3-diol, cyclohexylamine, tris(2-aminoethyl)amine, 2-amino-1-butanol, 2-amino-1-propanol, 2-aminoethanethiol, 1-phenylethylamine, benzylamine, and 2-pyridylethylamine, 5-norbornene-2-methylamine, and methacrylamide. In some embodiments, the secondary amine is dicyclohexylamine. Advantages of using primary and secondary amines such as these can include the ability to tune properties of the CPI.

The process can also include mixing the CPI with carbon dioxide (CO₂) to form a CPI−CO₂ adduct.

A CPI composition, an apparatus containing the CPI composition, a process of capturing CO₂ with the CPI composition, and a process of generating carbonate with the CPI composition are also disclosed.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a chemical reaction diagram illustrating a process forming cyclopropenimines (CPIs), according to some embodiments.

FIG. 2 is a chemical reaction diagram illustrating an experimental example of a process of forming a CPI.

FIG. 3A is a proton nuclear magnetic resonance (H¹ NMR) spectrum of a mixture of a CPI chloride product and hydrochloride (HCl) salt of dicyclohexylamine (DHA) formed in the process shown in FIG. 2.

FIG. 3B is an H¹ NMR spectrum of the HCl salt of DHA formed in the process shown in FIG. 2.

FIG. 3C is an H¹ NMR spectrum of the CPI chloride product formed in the process shown in FIG. 2.

FIG. 3D is an H¹ NMR spectrum of the CPI formed in the process shown in FIG. 2.

FIG. 4 is a chemical structure diagram illustrating a first set of CPIs a-o, according to some embodiments.

FIG. 5A is a chemical structure diagram illustrating CPIs with a series of R groups, according to some embodiments.

FIG. 5B is a chemical structure diagram illustrating CPIs with a series of R′ groups, according to some embodiments.

FIG. 6A is a chemical reaction diagram illustrating a process of forming a CPI-functionalized polymethacrylate, according to some embodiments.

FIG. 6B is a chemical reaction diagram illustrating a process of forming a CPI-functionalized polystyrene, according to some embodiments.

FIG. 6C is a chemical reaction diagram illustrating a process 620 of forming a CPI-functionalized polyurethane, according to some embodiments.

FIG. 7 is a chemical reaction diagram illustrating a process of CPI-facilitated CO₂ capture and transfer, according to some embodiments.

FIG. 8 is a diagram illustrating a sol-gel CO₂ capture/release process involving a triblock copolymer with CPI pendent groups, according to some embodiments.

FIG. 9 is a flowchart illustrating a process of forming CPIs, according to some embodiments.

While the invention is amenable to various modifications and alternative forms, specifics thereof have been shown by way of example in the drawings, and will be described in detail. It should be understood, however, that the intention is not to limit the invention to the particular embodiments described. Instead, the intention is to cover all modifications, equivalents, and alternatives falling within the spirit and scope of the invention.

### DETAILED DESCRIPTION

Embodiments of the present invention are generally directed to small-molecule synthesis and, more specifically, to synthesis of cyclopropenimine compounds. While the present disclosure is not necessarily limited to such applications, various aspects of the disclosure may be appreciated through a discussion of examples using this context.

Cyclopropenimines (CPIs) are a class of strong organic bases, which can act as enantioselective Brønsted base catalysts. CPIs can be used for direct-air-capture (DAC) of CO₂ by forming adducts with CO₂. Low-energy CO₂ release from the adducts can be accomplished with external stimuli (e.g., mechano- or photochemical activation). Further, CPIs can activate the captured CO₂ for subsequent chemical transformations, which can be followed by recovery of the CPI via basification. The aromatic stabilization and synthetic modularity of CPIs have enabled their use in catalytic transformations such as Michael additions, Mannich reactions, Wittig rearrangements, and ring-opening polymerization. Conjugate acids of CPIs can be utilized as Brønsted acid catalysts in reactions involving additions to oxocarbeniums and hydroamination of alkenes.

The ability to easily perform structural modifications and observe reactivity changes with CO₂ can facilitate development of a singular class of reagents for CO₂ transformations. A disadvantage of many organometallic systems or organic compounds (e.g., amidines, guanidines, phosphazenes, organoboranes) currently used for CO₂ activation is the difficulty in systematically modifying their steric and electronic properties. The synthetic modularity of CPIs may overcome these challenges.

CPIs exhibit unique properties that set them apart from other classes of compounds. The 2π-electron Hückel aromatic stabilization of the conjugate acid affords CPIs a much higher pKₐ than analogous organic bases. This may enable development of new catalyst platforms and transformations using CO₂ as a building block by providing insight into how CPIs react with CO₂, the stability of the resulting CPI−CO₂ adducts, and the reactivity of those adducts in common transformations involving CO₂.

Further, CPIs can be synthesized on a large scale from inexpensive substrates and processed into emulsions, membranes, particles, etc. that may be integrated within CO₂ reactors. Due to the unique solubility of CPIs, existing synthetic procedures have used dichloromethane as the solvent because is solvates the initial reagents and the final products. However, health risks associated with dichloromethane have led to increasing regulation of this solvent, making it undesirable and impractical for large-scale processes. However, attempts to replace DCM with other solvents, e.g., methyl tetrahydrofuran or chloroform, in current CPI synthetic procedures have not met with adequate success. Therefore, new techniques for forming CPIs that do not require a solvent in which both the reactants and products are soluble may be needed.

Embodiments of the present disclosure may be used to overcome these and other challenges. For example, disclosed herein are synthetic procedures for producing CPIs without solvents such as dichloromethane. This may allow fast, high-yield production of CPIs with a variety of structures/properties. In some embodiments, the CPIs may be synthesized on a large scale from readily available, inexpensive substrates and processed into emulsions, membranes, particles, etc., that may be integrated within CO₂ reactors. Such materials may enable DAC systems that operate at ambient conditions using localized, renewable energy sources. In some embodiments, the CPIs can be used in CO₂ transfer reactions resulting in formation of various polymers, small molecules, and metal carbonate salts.

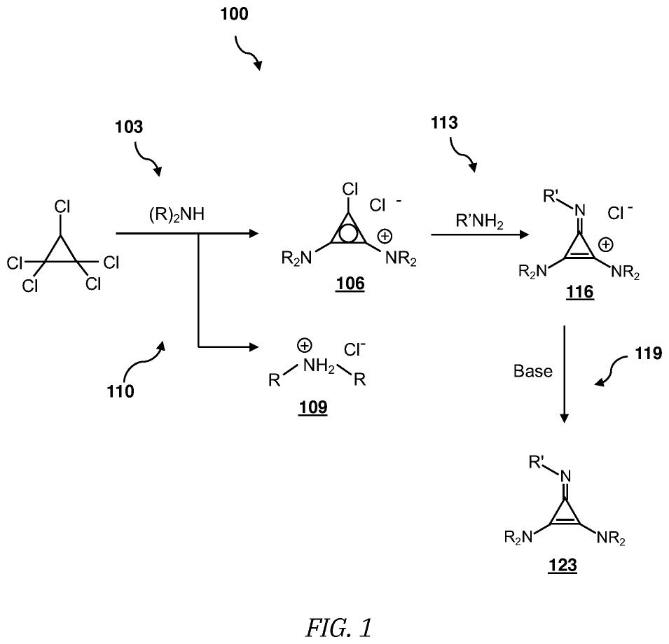

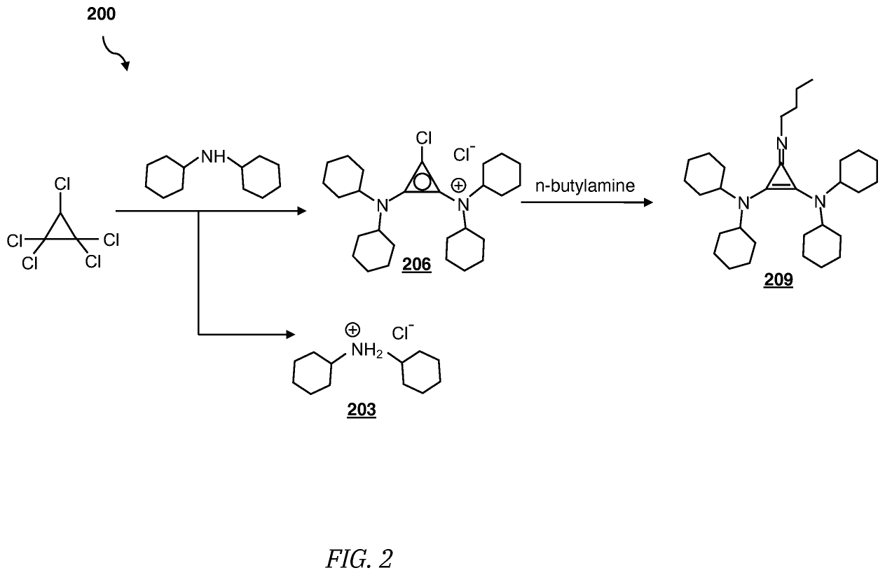

Referring now to the drawings, in which like numerals represent the same or similar elements, FIG. 1 is a chemical reaction diagram illustrating a process 100 of forming CPIs, according to some embodiments. In order to form the CPI, a solution of pentachlorocyclopropane (PCC) in ethyl acetate (EA) can be prepared. A secondary amine R₂NH (e.g.,) can be added to the PCC/EA solution. This is illustrated at operation 103. In some embodiments the secondary amine may be dicyclohexylamine (see, e.g., FIG. 2). However, a variety of secondary amines may be used, such as diethylamine, dipropylamine, diisopropylamine, dibutylamine, ditertbutylamine, diisobutylamine, morpholine, etc. The secondary amine may be added dropwise to minimize temperature evolution of the exothermic reaction. The reaction at operation 103 can result in precipitation of both a bis(amino)cyclopropenium chloride salt (“CPI−Cl salt”) 106 and an HCl salt of the secondary amine (“secondary amine salt”) 109. The precipitates 106 and 109 can be isolated (e.g., by filtration) and then dispersed in a solvent that will dissolve the CPI−Cl salt 106 without dissolving the secondary amine salt 109. An example of such a solvent is chloroform. The CPI−Cl salt 106 can then be separated from the secondary amine salt 109 by extraction of the CPI−Cl salt 106/solvent layer (e.g., extraction with water). This is illustrated at operation 110.

The isolated CPI−Cl salt 106 can be reacted with a primary amine (R′NH₂). This is illustrated at operation 113. Examples of primary amines that can be used may include, but are not limited to, n-butylamine, cyclohexylamine, propylimidazole, N,N-dimethyl-1-propanamine, N,N-dimethyldiethylenetriamine, 2-aminoethanol, 2-amino-1-propanol, 1,2-diaminoethane, 1-hydroxy-2-butylamine, 2-aminopropane-1,3-diol, cyclohexylamine, tris(2-aminoethyl)amine, 2-amino-1-butanol, 2-amino-1-propanol, 2-aminoethanethiol, 1-phenylethylamine, benzylamine, and 2-pyridylethylamine, 5-norbornene-2-methylamine, methacrylamide, 2-aminoethylmethacrylamide, 4-aminostyrene,

The CPI−Cl salt 106 and primary amine can be dissolved in a solvent insufficiently acidic for protonation of the final CPI product (CPI 123). An example of such a solvent is toluene, although other solvents may be used depending on the solubility of the primary amine and pKₐ of the solvent. The product of this reaction, a tris[amino]cyclopropenium (TAC) salt 116, can be washed with an alkaline solution. For example, to the toluene solution, an aqueous solution of about 1 mol/Liter (M) sodium carbonate (Na₂CO₃), about 1-2 M potassium carbonate (K₂CO₃), about 20-30% by volume (vol. %) ammonium hydroxide (NH₄OH), about 1 M sodium hydroxide (NaOH), etc. The aqueous alkaline solution can neutralize the TAC salt 116. This is illustrated at operation 119.

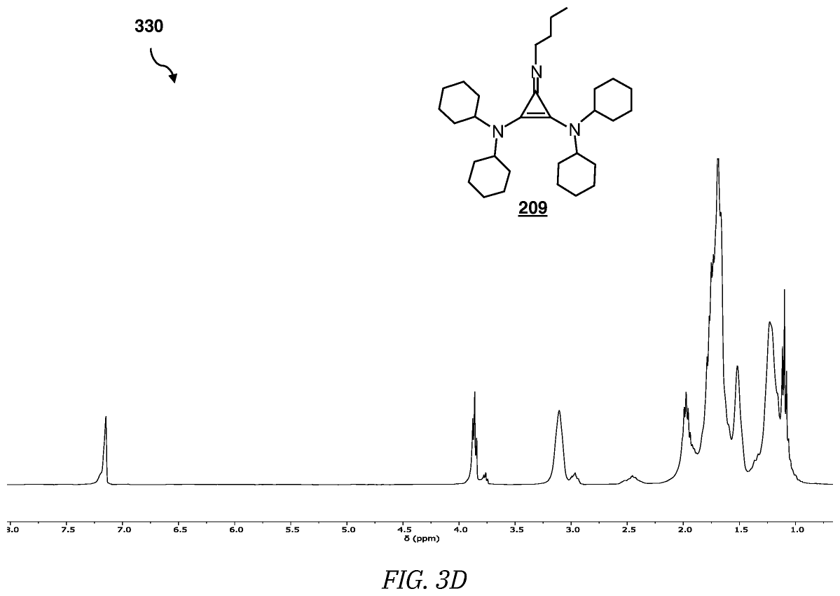

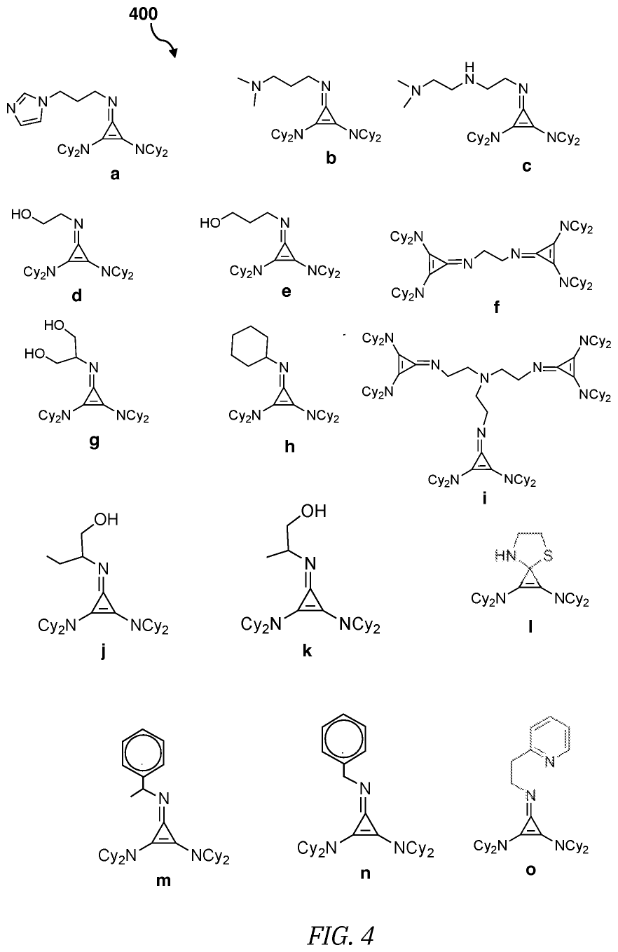

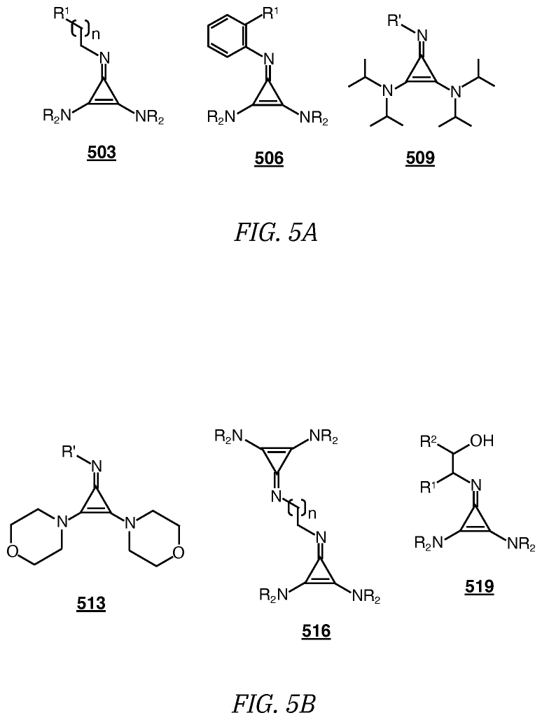

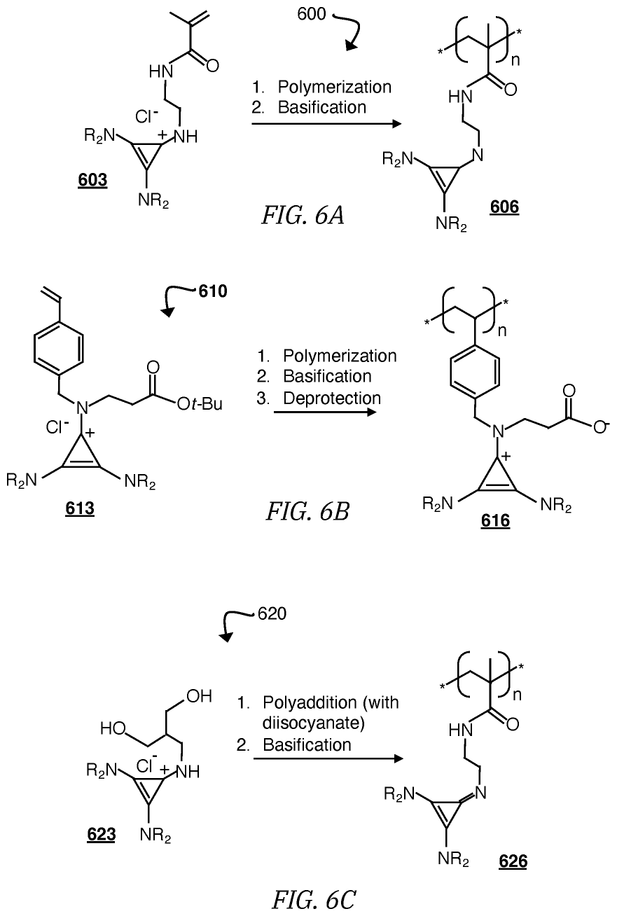

The TAC salt 116 formed at operation 119 can be a small molecule (see, e.g., FIGS. 4-5B). However, operation 119 may include polymerization (not shown) of the TAC salt 116 to form a polymer with TAC pendent groups (see, e.g., FIGS. 6A-6C) in some embodiments. For example, a primary amine where R′ is polymerizable (e.g., amines such as 2-aminoethylmethacrylamide, 4-aminostyrene, etc.) may be used at operation 113, resulting in a TAC salt 116 (“TAC monomer”) that may be polymerized at operation 119.

The neutralization (basification) of the TAC salt 116 (small molecule or polymer) at operation 119 can result in the corresponding small molecule (or polymer) CPI 123. The ability to vary R and R′ groups based on secondary and primary amine selection provides modularity to the CPI 123 core by allowing tuning of parameters such as reactivity, solubility, etc. Examples and effects of R and R′ groups are discussed in greater detail below.

FIG. 2 is a chemical reaction diagram illustrating an experimental example 200 of a process of forming a CPI small molecule, such as process 100 shown in FIG. 1. To an EA solution of PCC, dicyclohexylamine (DHA) was added dropwise over about an hour and then allowed to react for an additional hour (e.g., at operation 103 of process 100). This took place at room temperature while stirring. The temperature evolution during this reaction was minimal.

During the reaction, both a CPI chloride salt (“CPI chloride 206”) and a byproduct hydrochloride (HCl) salt of DHA (“DHA salt 203”) precipitated from the EA solution. These precipitates were isolated by filtration and rinsed 3× with EA. The isolated precipitates 206 and 203 were transferred to a flask and dispersed in chloroform for about 1 hour. CPI chloride 206 was found to be readily soluble, whereas the DHA salt 203 was not. The CPI chloride 206 was isolated by filtration from the majority of the DHA salt 203, and the chloroform layer was extracted with water in order to remove any remaining DHA salt 203 (e.g., at operation 110 of process 100). Isolated CPI chloride 206 was then reacted with n-butylamine to form a TAC salt (e.g., at operation 113 of process 100).

The TAC salt (not shown) was added to a basic aqueous solution (about 2 M NaOH) and stirred for about 1-2 hours to form CPI 209 (e.g., at operation 119 of process 100). The biphasic solution of CPI 209 resulting from the basification was added to a separatory funnel with toluene and additional 2M NaOH solution, and the mixture was vigorously shaken. After phase separation of the mixture in the separatory funnel, the toluene phase was removed. This toluene solution was dried using NaSO₄ and concentrated. Ethyl acetate was added to the concentrated solution to facilitate filtration of an off-white solid (CPI 209). The solid CPI 209 was rinsed with acetonitrile after filtration and then dried by heating under reduced pressure in a vacuum oven.

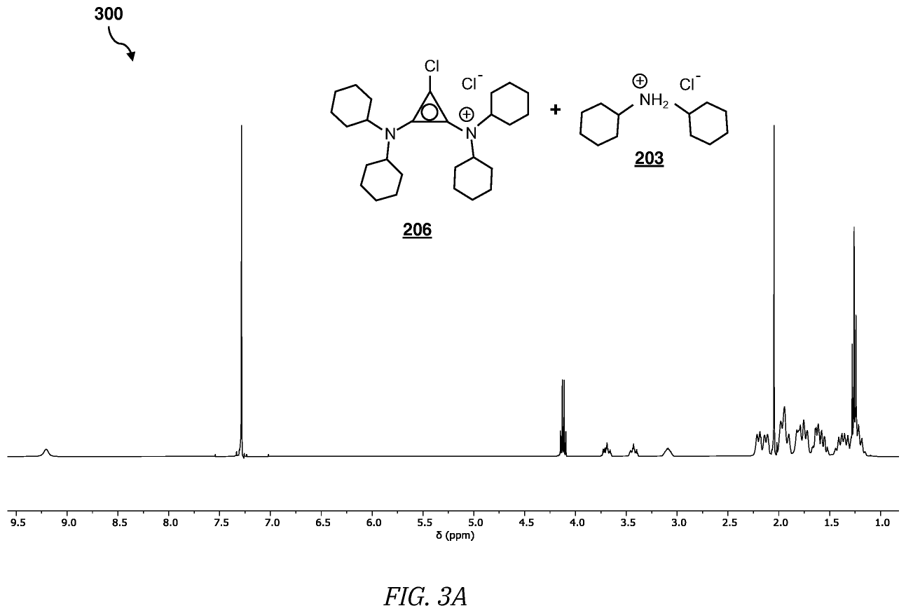

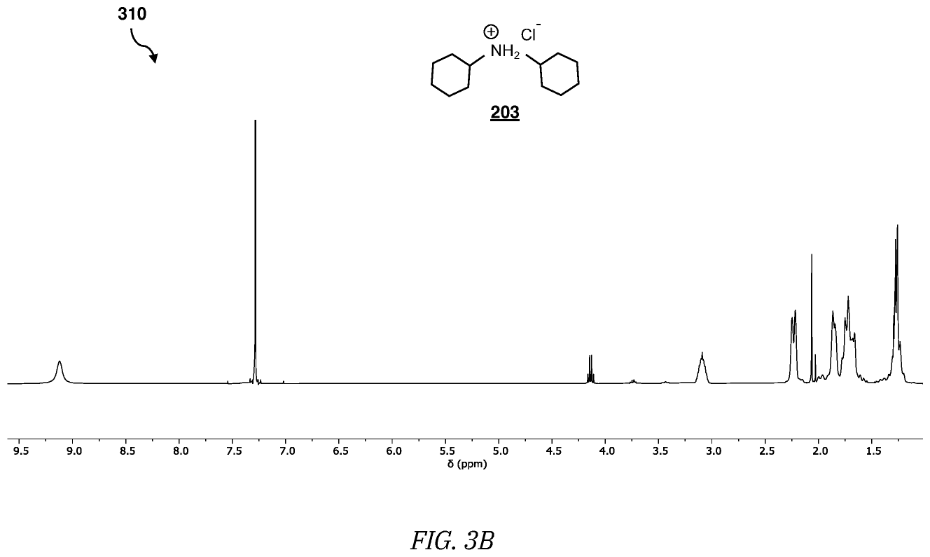

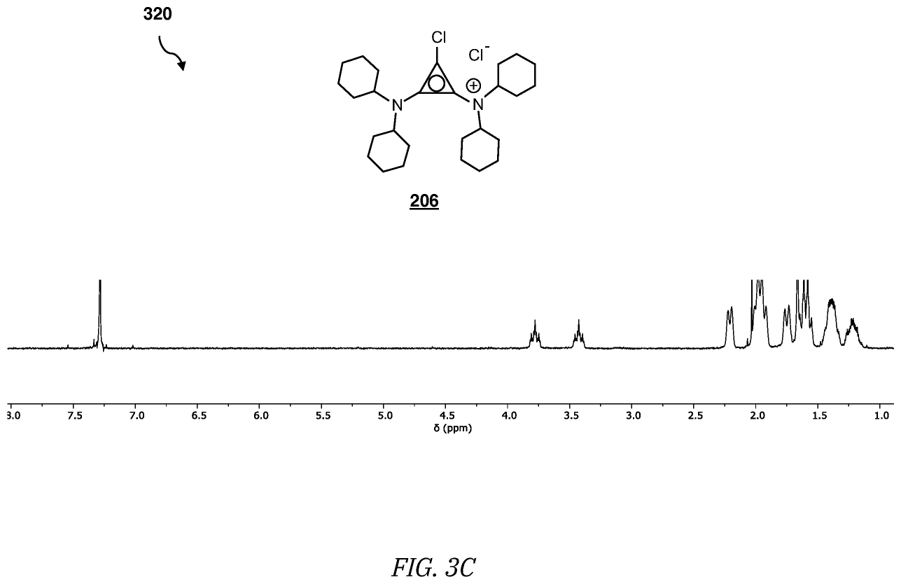

FIG. 3A is a proton (H¹) nuclear magnetic resonance (NMR) spectrum 300 of a mixture of the CPI chloride product 206 and HCl salt of DHA 203 formed in process 200. FIG. 3B is an NMR spectrum 310 of the isolated HCl salt of DHA 203 formed in process 200. FIG. 3C is an NMR spectrum 320 of the CPI chloride product 206 formed in process 200. NMR spectra 310 and 320 were obtained in deuterated chloroform. FIG. 3D is an H¹ NMR spectrum 330 of the CPI 209 formed in the process shown in FIG. 2. NMR spectrum 330 was obtained in deuterated benzene.

FIG. 4 is a chemical structure diagram 400 illustrating a first set of CPIs a-o according to some embodiments. These CPIs a-o may be formed in process 100 (FIG. 1) using DHA as the secondary amine. In order to form the different CPIs a-o, the primary amines used in process 100 can include propylimidazole (a), N,N-dimethyl-1-propanamine (b), N,N-dimethyldiethylenetriamine (c), 2-aminoethanol (d), 2-amino-1-propanol (e), 1,2-diaminoethane (f), 2-aminopropane-1,3-diol (g), cyclohexylamine (h), tris(2-aminoethyl)amine (i), 2-amino-1-butanol (j), 2-amino-1-propanol (k), 2-aminoethanethiol (l), 1-phenylethylamine (m), benzylamine (n), and 2-pyridylethylamine (o).

FIG. 5A is a chemical structure diagram showing CPIs 503-509 with a series of R groups, according to some embodiments. CPIs 503-509 illustrate further examples of CPIs that may be formed using process 100. The R groups can be modified to tune solubility, basicity, stability, etc. For example, CPI 503, which has morpholino R groups, may be more soluble than CPIs 506 and 509, which respectively have isopropyl and cyclohexyl R groups. Further, the R groups may be modified to induce twisting of the plane between the cyclopropene ring and the —NR₂ moieties due to steric interactions. This may impact the basicity and reactivity of the CPIs. The R′ group of CPIs 503-509 can be any appropriate organic substituent (see, e.g., FIG. 4).

FIG. 5B is a chemical structure diagram showing CPIs 513-519 with a series of R′ groups, according to some embodiments. CPIs 513-519 illustrate additional examples of CPIs that may be formed using process 100. The R′ groups of CPI 513 and CPI 516, respectively, include linear alkyl and cyclic aromatic moieties. The R′ group of CPI 519 includes an alcohol moiety. In CPIs 513-519, R¹ and R² can, independently, be hydrogen atoms or any appropriate reactive or unreactive functional groups. The R′ groups can be modified to tune interactions with CO₂. For example, when R′ includes an alcohol moiety (e.g., CPI 519 or CPIs 513/516 when R¹ has hydroxyl group), a synergistic effect on CO₂ capture analogous to alcohol-containing guanidine and amidine systems may be conferred. The R groups of CPIs 513-519 can be any appropriate organic substituents (see, e.g., FIGS. 5A and 5B).

In addition to the small molecule CPIs discussed with respect to FIGS. 4-5B, products of process 100 may be used to generate polymers with CPI and/or TAC ion pendent groups (see, e.g., FIGS. 6A-6C). In some embodiments, the polymers can be formed using polymerizable building blocks containing pendent CPIs or TAC ions. For example, TAC ion monomers such as those formed at operation 119 of process 100 (FIG. 1) may undergo polymerization and copolymerization by a variety of reversible deactivation radical polymerization (RDRP) reactions. In other embodiments, CPI and/or TAC ion pendent groups may be added post-polymerization. A wide variety of polymers may be synthesized with these pendent groups, such as polycarbonates, polystyrenes, polymethylmethacrylates, polymethacrylates, polyethers, polyesters, epoxide resins, polyamines, polyurethanes, etc.

FIG. 6A is a chemical reaction diagram illustrating a process 600 of forming a CPI-functionalized polymethacrylate, according to some embodiments. In this example, a methacrylate TAC monomer 603 is provided. The methacrylate TAC monomer can be formed, for example, at operation 119 of process 100 (where R′—NH₂ is 2-aminoethylmethacrylamide). The methacrylate TAC monomer 603 can be polymerized (e.g., via RDRP) and, following polymerization, neutralized to the free-base CPI. This yields a CPI polymer 606. While FIG. 6A illustrates a homopolymer 606, the methacrylate TAC monomer 603 can be copolymerized with other monomers, such as methacrylates having various functional groups. This can allow CPI reactivity with CO₂ to be tuned by utilizing the effects of neighboring pendent groups (e.g., hydroxyl groups).

FIG. 6B is a chemical reaction diagram illustrating a process 610 of forming a CPI-functionalized polystyrene, according to some embodiments. In process 610, a styrene/t-butyl ester TAC monomer 613 can be polymerized (1) to form a corresponding cationic TAC-functionalized polystyrene (not shown), which can be neutralized (2) by basification to yield a CPI pendent group. Deprotection (3) of the t-butyl ester functional group on the resulting CPI-functionalized polystyrene (not shown) can form the zwitterionic CPI-functionalized polystyrene 616. In other embodiments, non-zwitterionic CPI-functionalized polystyrenes may be formed by polymerization (1) of TAC-styrene monomers (e.g., formed using 4-aminostyrene at operation 113 of process 100) followed by basification (2).

FIG. 6C is a chemical reaction diagram illustrating a process 620 of forming a CPI-functionalized polyurethane, according to some embodiments. In some embodiments, amino-alcohols can be used to form TAC-diol monomers for organocatalyzed polyadditions, providing CPI-functionalized polyurethanes (e.g., CPI-functionalized polyurethane 626) following neutralization. For example, serinol can be reacted with a chloro-CPI to form the TAC-diol monomer 623 illustrated in FIG. 6C. Polyaddition with a diisocyanate can afford a corresponding TAC-functionalized polyurethane (not shown). Neutralization by addition of a base can result in the illustrated CPI-functionalized polyurethane 626.

Using polymerization techniques including those discussed with respect to FIGS. 6A-6C and/or other conventional synthetic methodologies, a variety of CPI-functionalized materials can be made using polymers and polymer networks with CPI pendent groups. Polymerization of TAC monomers may be accomplished as homogeneous solutions, as emulsions or suspensions, or in bulk. In some embodiments, cross-linking agents and/or other additives may be included in the polymerizations. Examples of materials formed using TAC monomers may include CPI polymer precipitates, crosslinked CPI polymer beads, high surface area CPI polymer foams (e.g., CPI-polyurethane foams), solid-phase resins (e.g., CPI-styrene resins), etc.

In some embodiments, the CPI polymers are incorporated into continuous CO₂ capture and transformation processes. For example, the CPI polymers can be multi-functional polymers for capturing CO₂ and either transforming the CO₂ into new chemicals or releasing it through external stimuli. Copolymerization of the CPI building blocks (e.g., TAC monomers or oligomers) with other functional monomers or oligomers and can be used to tune both CO₂ uptake and processability of the final polymers. Various macromolecular architectural considerations may also be used for tuning these properties. Examples of CPI polymer architectures may include linear, branched, dendritic, bottle brush, surface-grafted, etc. Techniques for automated polymerization, high-throughput characterization, predictive modeling, etc. may be employed to facilitate selection of material compositions. Through selection of monomers/oligomers used in these processes, both homogeneous and segmented morphologies can be generated, allowing control over air permeation, modulus, hydrophilic/hydrophobic balance, and other key structural features.

In some embodiments, the polymers with pendent CPIs and/or TAC ion derivatives can be processed in modular architectures such as particles, suspensions, membranes, gels, etc. CPI and/or TAC monomers/small molecules and oligomers may also be used to functionalize materials such as these in some embodiments. For example, nano- or microparticles can be formed with polymers having CPI pendent groups and/or be surface-functionalized with CPI molecules. CPI surfactants may be used as coatings as well. These and other CPI materials illustrated herein can be used to sequester, upcycle, and/or release CO₂ For example, CO₂ can be captured by CPI small molecules, polymeric pendent groups, etc.

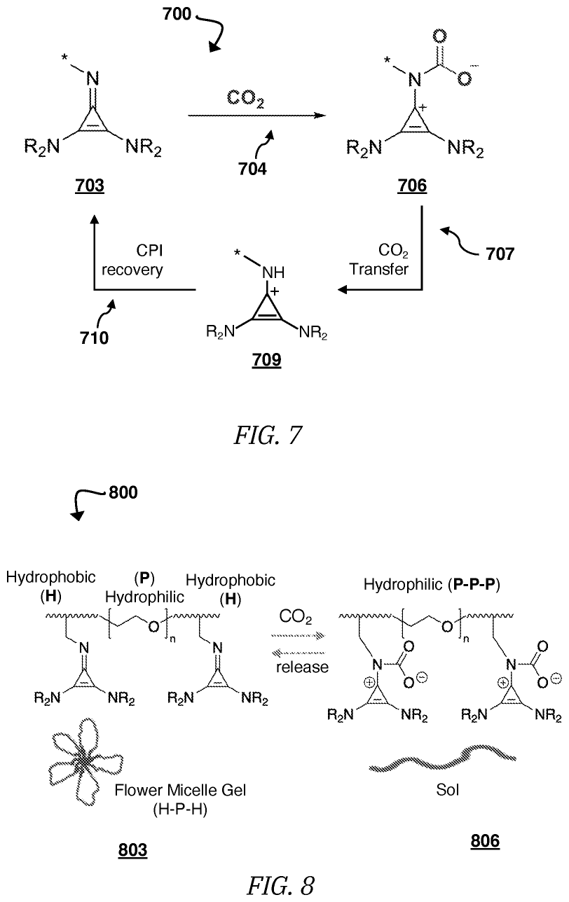

FIG. 7 is a chemical reaction diagram illustrating a process 700 of CPI-facilitated CO₂ capture and transfer, according to some embodiments. Process 700 can include reacting a CPI 703 with CO₂ gas to form a CPI−CO₂ adduct 706 (operation 704). This reaction may be carried out under ambient conditions in a polar organic solvent such as acetonitrile. CPI 703 may be any CPI small molecule or polymer formed by process 100 (FIG. 1) or a CPI derivative thereof. CPI 703 is illustrated with a starred bond, which can be a covalent bond, e.g., to a carbon atom of R′ (see, e.g., FIGS. 2 and 4-5B), a polymer/oligomer repeat unit (see, e.g., FIGS. 6A-6C), or a metal complex (not shown). In some embodiments, at least one R group may be replaced by a bond to a metal complex or polymer repeat unit as well. The CPI−CO₂ adduct 706 is a zwitterion characterized by an aromatic cyclopropenium (TAC) ion and an anionic carbonate moiety. In some embodiments, process 700 may be carried out on a large-scale and used for DAC and/or CPI-facilitated conversion of CO₂ into more useful chemical species.

The activated CO₂ may be released in a “CO₂ transfer” reaction (operation 707) resulting in a carbonate product (not shown) and a conjugate acid of the CPI (CPI−H⁺) 709. Compounds that react with carbonate to form products of the CO₂ transfer are not shown in FIG. 7. However, examples of CO₂ transfer reactions facilitated by CPI 703 can include reactions to form, e.g., carbonate minerals, cyclic carbonates, polyurethanes, polycarbonates, etc.

For example, in CPI-facilitated a mineralization reactions, the adduct 706 can be mixed with a metal halide (MX or MX₂), such as a metal chloride where M is sodium, potassium, magnesium, calcium, etc. This results in formation of a conjugate acid (CPI−H⁺ 709) halide (X⁻) salt and a bicarbonate (MHCO₃) or carbonate (MCO₃). For example, sodium bicarbonate (NHCO₃) can form when MX is NaCl, calcium carbonate (CaCO₂) can form when MX₂ is CaCl₂·2H₂O, and magnesium carbonate (MgCO₃) can form when MX₂ is MgCl₂.

In CPI-facilitated polymerizations, the CPI−CO₂ adduct 706 may act as a source of CO₂ for reactions to form polyurethanes or polycarbonates. For example, carbonate transfer from the CPI−CO₂ adduct 706 may facilitate cyclic carbonate/diamine routes to polyurethane formation, e.g., where the adduct 706 is reacted with 1,8-diamino-3,6-dioxaoctane and 1,4-bis(bromomethyl)benzene in acetonitrile to form the corresponding polyurethane and CPI−H⁺ 709 bromide. In another example, the CPI−CO₂ adduct 706 may be reacted with a polyethylene glycol and 1,4-bis(bromomethyl)benzene in acetonitrile to form a polycarbonate and CPI−H⁺ 709 bromide.

CPI-facilitated reactions involving the CO₂ transfer in process 700 may include formation of a variety of heterocyclic compounds. In some embodiments, catalytic ring-expansion of epoxides using CPI−CO₂ 706 can be used to generate 5-membered cyclic carbonates. The adduct 706 may be reacted with an epoxide in the presence of a co-catalyst (e.g., a halide salt) to form a cyclic 5-membered carbonate and a CPI−H⁺ 709 halide (CPI−H⁺/X⁻). For example, the CPI−CO₂ adduct 706 may facilitate conversion of glycidol to glycerol-1,2-carbonate. In other embodiments, the CPI−CO₂ adduct 706 may act as a CO₂ source in organocatalytic condensation reactions to form carbonates or carbamates. For example, the CPI−CO₂ adduct 706 may be reacted with an alcohol or amine and an organohalide to form a carbonate or a carbamate, respectively, and CPI−H⁺/X⁻. In further embodiments, the CPI−CO₂ adduct 706 may be reacted with 1-bromo-3-propanol to form trimethylene carbonate and CPI−H⁺/Br or reacted with 2-chloroethanol to form ethylene carbonate and CPI−H⁺/Cl⁻.

Additionally, the CPI−CO₂ adduct 706 may react with triphenyl silane (e.g., at room temperature) to catalytically produce triphenylsilyl formate. Additional reactions can be carried out using silyl formates to form synthetically useful compounds. For example, triphenylsilyl formate can react with an amine to produce a formamide species (e.g., converting piperidine to N-formylpiperidine). In further embodiments (not shown), CPI may react with CS₂ gas to form an adduct (CPI−CS₂) that may be used to form compounds such as dithiocarbamates and dithiocarbonates.

Upon loss of CO₂ by the CO₂ transfer of process 700, CPI 703 may be recovered (operation 710) by basification of CPI−H⁺ 709 (CPI−H⁺/X⁻). In some embodiments, addition of an aqueous base such as NaOH or NaNH₄ (e.g., about 1 M) to the CPI−H⁺/X⁻ solution can regenerate CPI 703. However, in other embodiments, the CPI recovery may be carried out under milder conditions using a biphasic process. In these instances, a solution of CPI−H⁺/X⁻ in a polar organic solvent (e.g., acetonitrile) can undergo solvent exchange from the polar organic solvent into a non-polar solvent (e.g., toluene, hexanes, combinations thereof, etc.). Addition of an aqueous alkaline solution, such as about 1 M Na₂CO_{3(aq.)} or about 20-30% vol. % NH₄OH_{(aq.)}, can then regenerate the CPI 703.

While not shown in FIG. 7, CPI-functionalized materials can provide various platforms for releasing captured CO₂ gas from the CPI−CO₂ adduct 706 in response to a stimulus. In some embodiments, CO₂ may be thermally released from the CPI−CO₂ 706. However, lower-energy release methods may be used as well. For example, the CPI-functionalized polymers may be used to form hydroxide-impregnated materials used for moisture-swing capture/release. Moisture-swing DAC uses water to preferentially displace CO₂ in the form of a carbonate and can have low energy-input when ambient heat is used to regenerate the dehydrated state of the polymer material.

In another example, photoredox catalysis may be used as a low-energy release mechanism of CO₂ through simple light-irradiation (e.g., with visible and/or ultraviolet light). In these instances, polymer CPI−CO₂ adducts can be irradiated in the presence of a photoredox catalyst, which can destabilize the adducts via single-electron transfer processes and cause release of the CO₂. This decarboxylation may also be used in tandem with photoredox-catalyzed carboxylation of various organic compounds.

Additionally, mechanical/mechanochemical force may be used to promote release of CO₂ from the CPI−CO₂ adducts and regeneration of the free-base CPI in some embodiments. For example, sonication of the polymer adducts can induce bond scission of labile N—C bonds between CPI and CO₂. Other techniques for N—C bond scission known to persons of ordinary skill may be used as well, such as appropriate chemical reactions.

In the transformation from CPI−CO₂ adduct to CPI+CO₂, the polymers change from a polar zwitterionic form to a non-polar neutral free-base form. This chemical change may be used to drive macromolecular phase transformations reversibly induced by CO₂ capture and release. For example, phase transitions of ABA triblock copolymers having hydrophobic A-blocks derived from CPI and a center B-block derived from hydrophilic polymers may be used. An example of this is illustrated in FIG. 8 (see below). These triblock copolymers can be prepared from difunctional hydrophilic blocks. In some embodiments, the R-groups on CPI can be used to drive phase changes. For example, morpholine-substituted CPIs are likely to be hydrophilic, and may therefore be used as the mid-block in order to increase the number of CO₂ capture sites within the entire triblock.

FIG. 8 illustrates a sol-gel CO₂ capture/release process 800 involving a triblock copolymer 803 with CPI pendent groups, according to some embodiments. As described above, the illustrated polymer 803 has a triblock ABA configuration with hydrophobic CPI A-blocks (H) and hydrophilic B blocks (P). The frustrated outer hydrophobic blocks fold back to form a hydrophobic pocket or “flower-like” micelle. Above ˜3-5% concentration in an aqueous solution, mild agitation can cause the hydrophobic chains to interdigitate, thereby connecting the micelles and producing a hydrogel. As shown in FIG. 8, CO₂ capture by passing air through the gel 803 can cause a phase-change from a gel to a sol 806. This is because the CPI−CO₂ adduct is hydrophilic and its formation causes unfolding of the micelles. The sol copolymer 806 can be captured and converted back to 803 upon release of the CO₂.

Various types of apparatus may be used in mediating absorption for DAC. For example, CPI-polymer materials for CO₂ capture/transfer may be employed in a packed bead reactor, trayed adsorption column, spray tower, spray dryer, etc. (see below). Techniques for gas-liquid mass transfer known to those of ordinary skill may be employed, and parameters such as flow rates, temperatures, concentrations, residence times, packing or tray types, nozzle design, droplet size (in spray methods) can be tuned.

In a packed bead reactor, there can be an absorption column that uses polymeric micro- and/or nanoparticles as a CPI-functionalized solid support resin. The absorption column can be packed with CPI-functionalized particles, and a CO₂-containing gas phase (e.g., atmospheric gas) can be passed through the column until CO₂ breakthrough is observed. Following the CO₂ exposure, the column can be detached, regenerated, and the gas released using techniques such as those discussed above (e.g., by photoredox/irradiation, mechanical force such as sonication, etc.). In some embodiments, CPI-functionalized particles are formed using CPI monomers (e.g., where R′ is a styrene moiety) behaving as surfactants. In these instances, polymerization with a core derived from a hydrophobic styrene and various concentrations of divinylbenzene (DVB), can generate highly crosslinked particles by mini-emulsion polymerization.

In a trayed absorption column, a CO₂-containing gas can be continually introduced at the bottom of the column while a CO₂-absorbing liquid, which includes a CPI-functionalized small-molecule or polymer solution, is introduced at the top of the column. As the gas and liquid phases mix in the column, the gas can percolate on trays positioned in the column to allow sufficient residence time for gas absorption into the liquid phase. The scrubbed gas can then be collected at the top of the column, and the CPI−CO₂-containing liquid can be collected at the bottom of the column for further downstream processing (e.g., including upcycling and/or release of the captured CO₂).

A spray tower can utilize an aqueous solution or emulsion of a CPI-functionalized polymer or CPI small molecules. This CPI-containing liquid can be sprayed from the top of the tower into a CO₂-containing gas. As in the trayed absorption column, the solution containing the captured CO₂ (CPI−CO₂ adducts) can then be collected at the bottom for further downstream processing.

A spray dryer can be similar to the spray tower. For example, a controlled mist of the CPI-containing liquid can be introduced into a tower or column concurrently with CO₂-containing air (e.g., heated CO₂-containing air). In this configuration, the liquid can be heated to ensure complete evaporation of the liquid phase and produce a solid aerosol and “wet” air. A cyclone separator can be used to disengage the solid material (the small-molecule or polymeric CPI−CO₂ adduct) from the flowing air.

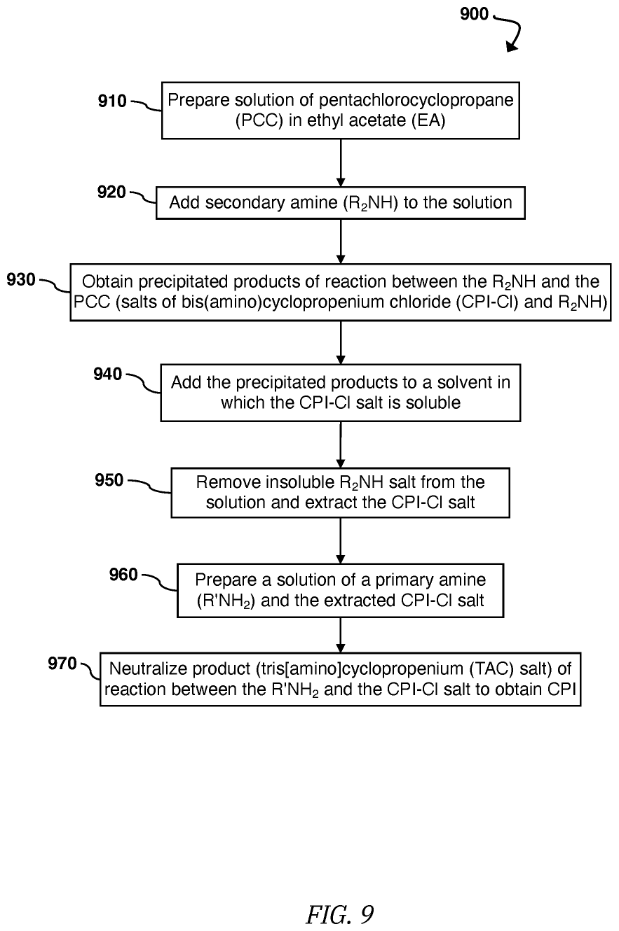

FIG. 9 is a flowchart illustrating a process 900 of forming CPIs, according to some embodiments. Process 900 may include operations and materials substantially similar to, or the same as, the operations and materials discussed with respect to FIG. 1. In process 900, a solution of PCC can be prepared in EA. This is illustrated at operation 910. A secondary amine R₂NH can be added to the PCC/EA solution. This is illustrated at operation 920. The secondary amine may be added dropwise to minimize temperature evolution of the exothermic reaction.

Products of a reaction between the secondary amine and PCC in the EA solution can be obtained. This is illustrated at operation 930. The products can include a CPI chloride salt and an HCl salt of the secondary amine (“secondary amine salt”), which can precipitate from the EA solution. At operation 930, the precipitates can be isolated (e.g., by filtration). The precipitates can then be dispersed in a solvent that will dissolve the CPI chloride salt without substantially dissolving the secondary amine salt. This is illustrated at operation 940. An example of such a solvent is chloroform. The insoluble secondary amine can be separated from the CPI chloride salt solution prepared at operation 940 by filtration. This is illustrated at operation 950. The CPI chloride salt can then be separated from any remaining secondary amine salt in the solution by extraction (e.g., extraction with water) of the CPI chloride salt/solvent layer.

The extracted CPI chloride salt can be reacted with a primary amine (R′NH₂). This is illustrated at operation 960. The CPI chloride salt and primary amine can be dissolved in a solvent insufficiently acidic for protonation of the final CPI product (CPI 123). An example of such a solvent is toluene, although other solvents may be used depending on the solubility of the primary amine and pKₐ of the solvent. The product of this reaction, a tris[amino]cyclopropenium (TAC) salt, can be washed with a basic solution to neutralize the TAC salt. This is illustrated at operation 970. In some embodiments, the TAC salt may be polymerized prior to neutralization (not shown in FIG. 9). The basification at operation 970 can result in a CPI product, such as the CPI 123 illustrated in FIG. 1.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus. Further, the word “providing” as used herein can refer to various actions such as creating, purchasing, obtaining, synthesizing, making available, etc. or combinations thereof.

As used herein, the articles “a” and “an” preceding an element or component are intended to be nonrestrictive regarding the number of instances (i.e., occurrences) of the element or component. Therefore, “a” or “an” should be read to include one or at least one, and the singular word form of the element or component also includes the plural unless the number is obviously meant to be singular.

As used herein, the terms “invention” or “present invention” are non-limiting terms and not intended to refer to any single aspect of the particular invention but encompass all possible aspects as described in the specification and the claims.

Unless otherwise noted, ranges (e.g., time, concentration, temperature, etc.) indicated herein include both endpoints and all numbers between the endpoints. Unless specified otherwise, the use of a tilde (˜) or terms such as “about,” “substantially,” “approximately,” “slightly less than,” and variations thereof are intended to include the degree of error associated with measurement of the particular quantity based upon the equipment available at the time of filing the application. For example, “about” can include a range of +8% or 5%, or 2% of a given value, range of values, or endpoints of one or more ranges of values. Unless otherwise indicated, the use of terms such as these in connection with a range applies to both ends of the range (e.g., “approximately 1-5 g” should be interpreted as “approximately 1 g to approximately 5 g”) and, in connection with a list of ranges, applies to each range in the list (e.g., “about 1-5 g, 5-10 g, etc.” should be interpreted as “about 1 g to about 5 g, about 5 g to about 10 g, etc.”).

As discussed above, CPIs and other compounds herein include R groups (e.g., R, R′, and RX, where x is an integer), which can be any appropriate organic substituent known to persons of ordinary skill. In some embodiments, the R groups can include substituted or unsubstituted aliphatic groups. As used herein, the term “aliphatic” encompasses the terms alkyl, alkenyl, and alkynyl.

As used herein, an “alkyl” group refers to a saturated aliphatic hydrocarbon group containing from 1 to 20 (e.g., 2 to 18, 2 to 8, 2 to 6, or 2 to 4) carbon atoms. An alkyl group can be straight, branched, cyclic, or any combination thereof. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted with one or more substituents or can be multicyclic as set forth below. Unless specified otherwise, the term “alkyl,” as well as derivative terms such as “alkoxy” and “thioalkyl,” as used herein, include within their scope, straight chain, branched chain, and cyclic moieties.

As used herein, an “alkenyl” group refers to an aliphatic carbon group that contains from 2 to 20 (e.g., 2 to 18, 2 to 8, 2 to 6, or 2 to 4) carbon atoms and at least one double bond. Like an alkyl group, an alkenyl group can be straight, branched, or cyclic, or any combination thereof. Examples of an alkenyl group include, but are not limited to, allyl, isopropenyl, 2-butenyl, and 2-hexenyl. An alkenyl group can be substituted with one or more substituents as set forth below.

As used herein, an “alkynyl” group refers to an aliphatic carbon group that contains from 2 to 20 (e.g., 2 to 18, 2 to 8, 2 to 6, or 2 to 4) carbon atoms and has at least one triple bond. Like an alkyl group, an alkynyl group can be straight, branched, or cyclic, or any combination thereof. Examples of an alkynyl group include, but are not limited to, propargyl and butynyl. An alkynyl group can be substituted with one or more substituents as set forth below.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cn-alkylthio, and heteroatom-substituted Cn-alkylthio. In some embodiments, lower alkylthios are contemplated.

The term “haloalkyl” refers to alkyl groups substituted with from one up to the maximum possible number of halogen atoms. The terms “haloalkoxy” and “halothioalkyl” refer to alkoxy and thioalkyl groups substituted with from one up to five halogen atoms.

As described herein, compounds of the present disclosure can optionally be substituted with one or more substituents, such as are illustrated generally above, or as exemplified by particular classes, subclasses, and species of the present disclosure. Each substituent of a specific group may further be substituted with one to three of, for example, halogen, cyano, sulfonyl, sulfinyl, carbonyl, oxoalkoxy, hydroxy, amino, nitro, aryl, haloalkyl, and alkyl. For instance, an alkyl group can be substituted with alkyl sulfonyl and the alkyl sulfonyl can be optionally substituted with one to three of halogen, cyano, sulfonyl, sulfinyl, carbonyl, oxoalkoxy, hydroxy, amino, nitro, aryl, haloalkyl, and alkyl.

In general, the term “substituted” refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a hetero cycloalkyl, can be bound to another ring, such as a cycloalkyl, to form a spiro-bicyclic ring system, e.g., both rings share one common atom. As one of ordinary skill in the art will recognize, combinations of substituents envisioned by this present disclosure are those combinations that result in the formation of stable or chemically feasible compounds.

Modifications or derivatives of the disclosed compounds are contemplated as being useful with the methods and compositions of the present disclosure. Derivatives may be prepared and the properties of such derivatives may be assayed for their desired properties by any method known to those of skill in the art. In certain aspects, “derivative” refers to a chemically modified compound that still retains the desired effects of the compound prior to the chemical modification.

The descriptions of the various embodiments of the present disclosure have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A process of forming a cyclopropenimine (CPI), comprising:
obtaining a first solution comprising pentachlorocyclopropane (PCC) dissolved a first solvent;
adding a secondary amine to the first solution;
obtaining precipitated products of a reaction between the secondary amine and the PCC in the first solution, the precipitated products comprising a CPI chloride (CPI−C1) salt and a secondary amine salt; and
mixing the precipitated products in a second solvent, wherein the CPI−Cl salt is substantially soluble in the second solvent, and the secondary amine salt is substantially insoluble in the second solvent.

2. The process of claim 1, further comprising:
extracting the CPI−Cl salt from the mixture; and
preparing a solution of the extracted CPI−Cl salt and a primary amine in a third solvent.

3. The process of claim 2, wherein the primary amine is selected from the group consisting of n-butylamine, cyclohexylamine, propylimidazole, N,N-dimethyl-1-propanamine, N,N-dimethyldiethylenetriamine, 2-aminoethanol, 2-amino-1-propanol, 1,2-diaminoethane, 1-hydroxy-2-butylamine, 2-aminopropane-1,3-diol, cyclohexylamine, tris(2-aminoethyl)amine, 2-amino-1-butanol, 2-amino-1-propanol, 2-aminoethanethiol, 1-phenylethylamine, benzylamine, and 2-pyridylethylamine, 5-norbornene-2-methylamine, and methacrylamide.

4. The process of claim 2, wherein the first solvent is ethyl acetate, the second solvent is chloroform, and the third solvent is toluene.

5. The process of claim 2, further comprising obtaining a tris[amino]cyclopropenium (TAC) salt formed in a reaction between the CPI−Cl salt and the primary amine.

6. The process of claim 5, further comprising neutralizing the TAC salt in an alkaline solution to obtain the CPI.

7. The process of claim 6, wherein the CPI has the following structure: 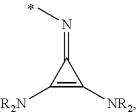
wherein each R is an organic substituent, and wherein the starred bond is to a carbon atom.

8. The process of claim 5, further comprising polymerizing the TAC salt.

9. The process of claim 8, further comprising neutralizing the TAC salt polymer in an alkaline solution to obtain CPI pendent groups.

10. The process of claim 1, wherein the secondary amine is dicyclohexylamine.

11. A composition comprising a cyclopropenimine (CPI), wherein the CPI is generated by a process comprising:
obtaining a first solution comprising pentachlorocyclopropane (PCC) dissolved a first solvent;
adding a secondary amine to the first solution;
obtaining precipitated products of a reaction between the secondary amine and the PCC in the first solution, the precipitated products comprising a CPI chloride (CPI−Cl) salt and a secondary amine salt; and
mixing the precipitated products in a second solvent, wherein the CPI−Cl salt is substantially soluble in the second solvent, and the secondary amine salt is substantially insoluble in the second solvent.

12. The composition of claim 11, wherein the process further comprises:
extracting the CPI−Cl salt from the mixture;
preparing a solution of the extracted CPI−Cl salt and a primary amine in a third solvent; and
obtaining a tris[amino]cyclopropenium (TAC) salt formed in a reaction between the CPI−Cl salt and the primary amine.

13. The composition of claim 12, wherein the first solvent is ethyl acetate, the second solvent is chloroform, and the third solvent is toluene.

14. The composition of claim 12, wherein the process further comprises neutralizing the TAC salt in an alkaline solution to obtain the CPI.

15. The composition of claim 12, wherein the primary amine is selected from the group consisting of n-butylamine, cyclohexylamine, propylimidazole, N,N-dimethyl-1-propanamine, N,N-dimethyldiethylenetriamine, 2-aminoethanol, 2-amino-1-propanol, 1,2-diaminoethane, 1-hydroxy-2-butylamine, 2-aminopropane-1,3-diol, cyclohexylamine, tris(2-aminoethyl)amine, 2-amino-1-butanol, 2-amino-1-propanol, 2-aminoethanethiol, 1-phenylethylamine, benzylamine, and 2-pyridylethylamine, 5-norbornene-2-methylamine, and methacrylamide.

16. The composition of claim 11, wherein the CPI has the following structure: 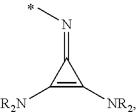
wherein each R is an organic substituent, and wherein the starred bond is to a carbon atom.

17. The composition of claim 16, wherein the CPI is a pendent group on a polymer.

18. A process of carbon dioxide (CO₂) capture, comprising:
providing a composition comprising a cyclopropenimine (CPI), wherein the providing comprises:
obtaining a first solution comprising pentachlorocyclopropane (PCC) dissolved a first solvent;
adding a secondary amine to the first solution;
obtaining precipitated products of a reaction between the secondary amine and the PCC in the first solution, the precipitated products comprising a CPI chloride (CPI−Cl) salt and a secondary amine salt; and
mixing the precipitated products in a second solvent, wherein the CPI−Cl salt is substantially soluble in the second solvent, and the secondary amine salt is substantially insoluble in the second solvent.

19. The process of claim 18, wherein the providing further comprises:
extracting the CPI−Cl salt from the mixture;
preparing a solution of the extracted CPI−Cl salt and a primary amine in a third solvent;
obtaining a tris[amino]cyclopropenium (TAC) salt formed in a reaction between the CPI−Cl salt and the primary amine; and
neutralizing the TAC salt in an alkaline solution to obtain the CPI.

20. The process of claim 18, further comprising mixing the composition with the CO₂ to form a CPI−CO₂ adduct.

21. A process of generating carbonate, comprising:
providing a composition comprising a cyclopropenimine (CPI), wherein the providing comprises:
obtaining a first solution comprising pentachlorocyclopropane (PCC) dissolved a first solvent;
adding a secondary amine to the first solution;
obtaining precipitated products of a reaction between the secondary amine and the PCC in the first solution, the precipitated products comprising a CPI chloride (CPI−Cl) salt and a secondary amine salt; and
mixing the precipitated products in a second solvent, wherein the CPI−Cl salt is substantially soluble in the second solvent, and the secondary amine salt is substantially insoluble in the second solvent.

22. The process of claim 21, wherein the providing further comprises:
extracting the CPI−Cl salt from the mixture;
preparing a solution of the extracted CPI−Cl salt and a primary amine in a third solvent;
obtaining a tris[amino]cyclopropenium (TAC) salt formed in a reaction between the CPI−Cl salt and the primary amine; and
neutralizing the TAC salt in an alkaline solution to obtain the CPI.

23. The process of claim 22, further comprising reacting the composition with carbon dioxide (CO₂) to form a CPI−CO₂ adduct.

24. The process of claim 23, further comprising:
obtaining, from the CPI−CO₂ adduct, the carbonate and a conjugate acid of the CPI;
recovering the CPI from the conjugate acid of the CPI by mixing the conjugate acid of the CPI with an alkaline solution selected from the group consisting of an aqueous solution of Na₂CO₃ and an aqueous solution of about 20-30 vol. % NH₄OH.

25. An apparatus, comprising:
a first component configured to provide a composition for capturing carbon dioxide (CO₂), wherein the composition comprises a cyclopropenimine (CPI) generated by a process comprising:
obtaining a first solution comprising pentachlorocyclopropane (PCC) dissolved a first solvent;
adding a secondary amine to the first solution;
obtaining precipitated products of a reaction between the secondary amine and the PCC in the first solution, the precipitated products comprising a CPI chloride (CPI−Cl) salt and a secondary amine salt; and
mixing the precipitated products in a second solvent, wherein the CPI−Cl salt is substantially soluble in the second solvent and the secondary amine salt is substantially insoluble in the second solvent.
